(19) United States
(12) Patent Application Publication
(10) Pub. No.: US 2015/0224295 A1
(43) Pub. Date: Aug. 13, 2015

(54) THERAPEUTIC DEVICE AGAINST CANCER SPREAD AND METASTASIS

(71) Applicant: Fusheng LI, Qingdao, Shandong (CN)

(72) Inventors: Fusheng Li, Qingdao (CN); An Li, Qingdao (CN); Jing Du, Qingdao (CN); Xiaomei Yang, Qingdao (CN)

(21) Appl. No.: 14/424,710

(22) PCT Filed: Aug. 12, 2013
(86) PCT No.: PCT/CN2013/000949

§ 371 Date: Feb. 27, 2015

(30) Foreign Application Priority Data
Aug. 30, 2012 (CN) 201210313291.0
Aug. 30, 2012 (CN) 201220434659.4

(51) Int. Cl. A61M 37/00; A61H 7/00; A61N 1/36; A61H 23/02

(52) U.S. Cl. CPC A61M 37/0092; A61H 23/0245; A61H 7/005; A61H 7/007; A61N 1/36; A61H 2201/0207; A61H 2201/10; A61H 2201/1215; A61H 2201/1671; A61H 2201/1695; A61N 5/00

(57) ABSTRACT

A therapeutic device against cancer spread and metastasis specifically integrates recovery therapy with operative chemo-radiotherapy and is used for prevention and treatment of cancer spread and metastasis. This is a device comprising multifunctional friction heads (1) coupled to low and medium frequency DC, a housing (2) with a motor mounted therein, a switch (3), a handle (4), and an integration of laser, ultrasound, microwave, radiofrequency, and medicine intervention. By evenly cooperating medication massage, electrically stimulation, deep heating, medication injection and anticancer medicine, the therapeutic device can recover function of lesion region and prevent cancer cell spread.

### TECHNICAL FIELD

The present invention pertains to a medical device, particularly a therapeutic device which combines massage and medications with physical therapy to treat cancer spread and metastasis.

### BACKGROUND ART

Modern medical science hopes to cure cancer through early detection and early treatment. But 90% of patients with advanced cancer died from cancer spread and metastasis. People still do not know the cause of cancer spread and metastasis. Once the cancer has diffused, the doctors are only able to slow down the pace of development but cannot really cure it. Numerous domestic and foreign experts and professors have put forward many ideas and opinions on this. They are summarized as follows.

On Aug. 28, 2006, Reference News reprinted a report by the New York Times, Scientists Understand Cancer Spread, originally published on Aug. 15, 2006. The article held that “the environment of the organ being invaded, the micro-environment, plays a key role in cancer metastasis”. The idea that the micro-environment plays a key role sounds right, but not without one-sidedness. The argument has simply turned from emphasizing the key role of cancer cells proliferation to the function of the micro-environment.

On May 5, 2005, Reference News reprinted a report by USA Science Daily website. Studies have shown that the main cause of tumor spread may be cell hypoxia. Once confirmed, it may subvert the traditional cancer treatment modalities. The prevailing view is that gene mutations cause cancer to spread.

Cancer: The Evolutionary Legacy, by Mel Greaves of the UK, Century Publishing Group, Shanghai Science and Technology Press, December2010, the first edition. Chen Zhu, the Minister of Health and an Academician, wrote in the preface “Cancer has been a serious threat to human health and life”. Why is it so difficult to achieve satisfactory results from various cancer treatments? Since the 1970s, with the development of molecular biology and genomic technology, scientists have made new breakthroughs in their research into pathogenetic mechanism and related oncogenes from a microscopic standpoint. However, a unified macro understanding of the cancer has always been in lack. Why do cancer treatments fail? This is because cancer treatment is a double-edged sword that not just kills cancer cells but also bring more selection pressure, prompting new mutations to occur in a way to “help” the development of cancer cells.

Krankheit als Weg, by T. Dethlefsen and R. Dahlke, Chunfeng Literature and Art Publishing House, Sep. 1999, the first edition (The theoretical understanding of the disease and treatment on page 4). “We'll see by observing that modern medicine's most exciting discussion and development are limited to a variety of methods and functions, and they rarely come to the theory and philosophy of medicine. Reasons for the failure of modern medicine are not its methods but its understanding of the whole world. Its methods are often silently and unconsciously built on the basis of such an understanding. The failure of modern medicine lies in the lack of a philosophy.

Cancer Is Just A Chronic Disease, by Professor He Yumin, Shanghai Science and Technology Press, August 2008, the first Edition. Professor He wrote in his book (see page 4, page 12, page 43) “we just improve early treatment of cancer and still don't have much effective cure for advanced cancer”. The famous American oncologists Douglas Hanahan has summarized it as “The road from the past goes nowhere, and we look forward to an entirely different research method. Admittedly, this change depend primarily on the progress of technology, but it also depends on the update of the most fundamental concepts.” Scientists have studies 500 cases of “self-healing” cancer patients reported around the world in the past 100 years and found that the patient's physiological and metabolic environment have undergone changes not conducive to the growth and reproduction of cancer cells. The root cause of malignant cells tells us that the self-regulation and repair of metabolic environment is the real cure for cancer. If we can use artificial means to “reproduce” this good luck to all cancer patients, we will open up a promising shortcut for the human to defeat cancer.

Current Technology Cancer Therapy Machine, National Invention Patent ZL200610069987.8, approved on Jun. 29, 2011. Japanese Patent No. 5047301, approved on Jul. 27, 2012. Principles and methods of treatment of major diseases have been proposed in the patent description. The principles hold that symmetric and equivalent combination of both the structure and function, rather than one of them, plays a decisive role, which is the essence of major diseases. Cancer spread and metastasis has been discussed in detail in the patent description.

### 1. Function Recovery

Changes in the overall function restrain normal cells and promote its transformation into cancer cells. They may also restrain cancer cells and promote its transformation into normal cells. Normal cells decreasing as a result of dysfunction and cancer cells proliferating due to structural damage are the two components of the overall symmetry. They are two sides of the same issue. The lack of one side denies the existence of the other. There is no major or minor side. The structure does not determine the function. Inhibiting cancer cell proliferation does not mean an increase in the surrounding normal cells. Function recovery treatment can not only eliminate the side effects from surgery, radiotherapy and chemotherapy but also guarantee the normal treatment of the damaged cells. It basically eliminates the recurrent basis of cancer cells, reverse the direction and trend in the development of cancer, and significantly reduce mortality.

### 2. Cancer Spread and Metastasis

When cancer cells grow into lymphatic or blood vessels, cancer spread and metastasis occurs. The living environment surrounding cancer cells is a dysfunctional micro-circulation environment featuring overall ischemia and hypoxia. The degree of interaction between the cancer cells and the direct environment is the key for cancer spread and metastasis. With current technology, the surgery may remove the cancer cells constrained in their function and in the dormant stage. But it often will not be able to treat cancer. As anti-metastatic mechanism of the patients also disappears, the balance is broken, with cancer metastasis being an unavoidable result. Direction and location of cancer spread and metastasis is based on the lesion of the anatomical location in the structure, lymph and blood conduction, the surrounding functional decline, and suitable environment for the living and development of cancer.

### 3. Harms Caused by Cancer Spread and Metastasis

In the anti-injury treatment of surgery, radiotherapy and chemotherapy, although symptoms of the disease get temporarily relieved, the overall function around the lesion declines. Normal cells development is inhibited by a new blow instead of being restored. The treatment completely destroys the possibility of restoring overall function around the lesions. As the functions around the lesions in patients with advanced cancer are mostly at the organ level or system level with a wide range of impacts, this new blow is fatal. The patients have lost the foundation for long-term survival. The trend of death has been set. Large number of clinical cases have proved that the direct cause of death in the majority of cancer patients is not cancer itself, but the decrease in the survival function.

If the patient's metabolic function still exists, it will not generate an external environment appropriate for cancer metastasis. It also will not inhibit the mechanism preventing the proliferation, spread and metastasis of cancer cells which result in the overall lethal effect. Although seriously ill, the patients will not die. This is also the root cause in that the advanced cancer patients receiving anti-injury treatment which kills cancer cells without a recovery therapy tend to live no more than three to five years.

### 4. Treatment of Cancer Spread and Metastasis

In the case of acute infectious diseases or laboratory mice implanted with cancer cells, the main pathogenic factors are external and can be quickly cured. In contrast, cancers and cardiovascular diseases are chronic, resulting from prolonged energy consumption without resupply and thus causing damage to structures. The disease cannot be cured quickly while the patients will not perish quickly. Injury therapy aiming to kill cancer cells on the basis of significantly impairing function is a short-term radical surgery at the cost of life and will not succeed. Cancer spread and metastasis is a result of the interaction between cancer cell proliferation and micro-environment dysfunction. Killing cancer cells or restoring micro-environment function also cannot stop cancer spread and metastasis. To stop and reverse cancer spread and metastasis, the key factor is to provide an equivalent and overall treatment combining injury therapy with micro-environment function restoration. The new treatment can prevent cancer proliferation from happening. Even if it does proliferate, it can be cured. The advanced treatment technology combining both physical and chemical factors not only restores function while providing injury treatment, but also prevents and cure cancer spread and metastasis. The treatment time is significantly shorter than that of the existing technologies.

Cancer Therapy Machine in prior art also has the following disadvantages.

(1). Although the Cancer Therapy Machine in prior art follows the principle of combining structure and function and refers to the symmetric and equivalent interaction at different levels as the nature of the disease, it does not fully realize the number of changes between the primary and secondary levels of interaction between structure and function. It cannot locate the positions of cancer spread and metastasis and their relationships. Cancer spread and metastasis is the result of functional decline and structural damage at system level at the primary and secondary levels of interaction. As for classification in terms of prevention and treatment of cancer spread and metastasis, causes, levels and sites of spread and metastasis site are more important than symptoms. Only after clarifying the lesion sites and their level can people develop the right treatment for therapeutic purposes.

(2). For the treatment by mechanical movement of Cancer Therapy Machine in prior art, the DC squeezing friction head and mid or low-frequency static friction head function separately. The target is decentralized rather than being focused upon. They do not follow a unified synergistic therapeutic procedure going from low level to high level to improve the therapeutic effect.

(3). Cancer Therapy Machine in prior art lacks laser treatment factors, thus lacking accuracy and persistence in treatment. It cannot meet the requirements for enhanced treatment of cancer spread and metastasis.

### CONTENTS OF INVENTION

Philosophy is the study of the theoretical basis and general law for the cause, development and metastasis of disease and illness. The starting point is the unity of opposites. The opposites in the unity are in completely symmetrical interdependence. The two sides are at the same level in nature. But their power difference defines them as primary and secondary side. Restrained by the unity, the scope and means of power difference are limited. Different holistic understanding of the disease leads to different treatment orientation and treatment outcomes. Modern medicine holds that cancer is a local manifestation of systemic disease. As structure determines function, the conflict between structural damage and damage resistance forces determines disease outcomes. One shall use surgery, radiotherapy and chemotherapy to kill cancer cells based on anti-injury approach. The treatment and concepts above are not without one-sidedness. The following are the major reasons.

1. In terms of cancer awareness: Local lesions and systemic disease should be in symmetrical interdependence. Traditional Chinese medicine theory believes that the yin and yang should be in balance. However, local lesions come in different levels and positions, such as cells level lesions, organizational level lesions, organ level lesions, and system level lesions. Among them, only the system-level lesions can be interpreted as a local manifestation of systemic disease. The two sides are in symmetrical interdependence which cannot be explained as the relationship between systemic diseases and local lesions at other levels. Thus, the cancer awareness of modern medicine, that is, local manifestations of systemic disease, is lack of specificity and cannot define the nature and characteristics of systemic disease. It is unable to determine the level and position of local manifestations. It cannot explain the interaction between local manifestation and systemic disease. It cannot guide clinical treatment.

2. In the treatment of cancer: Cancer results from the interaction between different attributes at different levels. Structural properties and functional properties are in symmetric and balanced interdependence. Power difference between the two sides defines them as the primary and secondary side. The power of a structure determines its own function and development. It cannot determine the function and development of the structures around it. Modern medicine using surgery, radiotherapy and chemotherapy to kill cancer cells based on anti-injury approach is going in the wrong direction. Restrained by the diseased functions around symmetric lesions, the therapy has a huge side effect, causing cancer spread and metastasis.

Chinese medicine believes in correspondence and harmony between man and nature, strengthening body resistance and eliminating evil, treating both manifestation and root cause of disease, and a balance of yin and yang. This holistic treatment principle based on ancient philosophy has been proved by thousands of years' practice to be right and in line with the principles of nature. It can develop by itself and also combine with the Western medicine. At present, the overall understanding of the disease has not been taken as a general idea of physiology, pathology, and cause and has not been implemented in clinical diagnosis and treatment. In modern medicine, the universality and specificity, same level and different levels, micro and macro understanding, internal and external factors, gradient and mutation, quantitative changes and qualitative changes, structural and functional lesions, and physics and chemistry have not been taken as a unified whole. Instead, the therapists simply focus on one side of structural damage. They provide partial treatment to pursue overall efficacy and use basic technology to achieve the effects of advanced technology. The main method of treatment is surgery, chemotherapy and other anti-injury treatment. And a lack of recovery treatment will restrict and offset anti-injury treatment. No matter how the anti-injury treatment develops and improves itself through increasing treatment efforts and increasing the cost of treatment, it can only play a limited role and will not produce the overall effect of curing. This is why we still do not know the cause of the disease, leading to treatment in the wrong direction. The treatment cannot reduce morbidity and mortality. There is no cure and treatment costs are going increasingly higher.

The present invention, based on the theory that the mutual restraint of system level functional lesions and system level structural lesions is the overall cause of cancer spread and metastasis, integrates the squeezing friction head and the static friction head into a multi-functional friction head providing medical massage. A speed and pressure controller allows for shallow and deep therapeutic treatment to improve the mechanical massage effect. The multi-functional friction head works cooperating with the function of DC low and mid frequency electro-therapy, laser, ultrasonic wave, microwave, radio frequency(RF), injecting drug intervention, and anti-cancer drugs. Through generating symmetric interaction, it cures system level functional lesions. It forms a mutually reinforcing combination with surgery-based chemotherapy to kill cancer cells to the overall effect of removing the root cause. Overall treatment of the root cause can produce a significant effect on the primary and surrounding lesion of cancer cells at organ levels. It may prevent cancer spread and metastasis and eliminate the side effects of treatment. Overall treatment of cancer spread and metastasis lesion at system level can control and cure cancer spread and metastasis.

In order to fully disclose the invention and manufacturing process, the present invention is to introduce in detail the principles and techniques of the treatment of cancer spread and metastasis.

### 1. An Innovative Treatment Principle Regarding Cancer Spread and Metastasis

The present invention believes that the lack of symmetric and equivalent recovery function is the root cause of cancer spread and metastasis and the final death. Through holistic treatment of the root cause, one may eliminate spread and metastasis and significantly reduce mortality. The patent specification of Cancer Therapy Machine has proposed a major principle and method of treating cancer. The only way to cure cancer is to present an innovative basic medical theory and clinical treatment technology in accordance with the law of nature. Based on this theory and technology, the present invention provides an innovative treatment principle for cancer spread and metastasis as follows.

### (1) On Cancer Etiology and Treatment

Human body is an interdependent and interactive whole of symmetric and equivalent structures and functions. Cancer is an overall lesion from symmetric and mutual restraint between localized structural damage and overall functional decline at different levels. Symmetric and mutual restraint between DNA structural damage and cell dysfunction produce precancerous lesions. The cancer is caused by symmetric and mutual restraint between structural lesion at cell level and functional decline at tissue level. Modern medicine defines disease as lesions at the same level and believes that structure determines function. The therapists favor partial treatment featuring surgery, chemotherapy and anti-injury treatment, supplemented by function restoration. Restrained by symmetric and overall functional decline, it cannot cure cancer with overall lesions.

Therapeutic device against cancer spread and metastasis combines targeted recovery function with surgery, chemotherapy and anti-injury treatment in a symmetric, equivalent and mutually reinforcing way to cure cancers.

### (2) On Cancer Treatment Technology

Human life movement is an advanced form of movement based on interdependent and mutually reinforcing primary physical and chemical movement. Cancer is a lesion of the advanced form of movement based on symmetrically and mutually restrained physical and chemical lesion. Technological products in the field of modern medical treatment are not based on the law of the movement of life. It is mainly based on modern science in which physics and chemistry play a separate role. It is a primary treatment technology which, restrained by symmetrical lesions, is hardly effective and with serious side effects. Currently, the world's patents on chemical anti-cancer drugs has reached over one thousand. However, no anticancer drug has recognized effects. Cancer's resistance to drugs has caused 90% of metastatic cancer treatment to fail. A variety of radiotherapy equipment such as radioactive cobalt, gamma knife, and form and intensity modulation, and physical therapy such as laser, ultrasound focus, microwave, and radio frequency can expand the range of treatment, reduce the side-effects and provide palliative care. But they have little effect on improving cancer cure rate. Therapeutic device against cancer spread and metastasis, based on the law of life movement, combines physics and chemistry in a symmetric, equivalent and mutually reinforcing way and thus obtain an advanced treatment technology. It is much more effective than the primary treatment technology. It is specialized in treating cancer lesions in the form of advanced movement. It can be developed combined into thousands of advanced treatment techniques for critical illness to meet treatment needs.

### (3) On Cancer Prevention, Treatment and Testing

a. Cancer is evolved from general and common diseases. As long as the function surrounding the structural damage remains normal, there is no risk of cancer. Through targeted recovery (by using the therapeutic device three minutes a day), one can prevent cancer. Relying solely on improving lifestyle lacks specificity and can prevent cancer.

b. The overall treatment of cancer is to treat both manifestation and root cause of disease. Through treating structural lesions at tissue level and functional decline at organ level in an equivalent and mutually reinforcing way, it promotes treatment effects on cancer patients at medium and advanced stages. Through treating structural lesions at organ level and functional decline at system level in an equivalent and mutually reinforcing way, it promotes treatment effects on cancer patients at the advanced stage. Through treating structural damage at system level and functional decline at system level in an equivalent and mutually reinforcing way, it promotes treatment effects on patients with cancer spread and metastasis. Clinical data show that 90% of patients with medium or advanced cancer did not receive treatment against functional decline around the lesions. Resultantly, within a year after surgery, radiotherapy and chemotherapy, cancer cells began to spread and transfer. So one must adopt the overall treatment. In the overall treatment of cancer, surgery, radiotherapy and chemotherapy no longer play a major role. Instead, they become the equivalents. Recovery therapy, through targeted positioning and use of advanced treatment technology, rises into peer status from the secondary position. Through mutual reinforcement and interaction, the two sides treat the root cause and eliminate spread and metastasis to cure the cancer. The treatment adopts both structural and functional standards to improve the level of treatment and to cure cancer.

### (4). Cancer Patients' Mortality

Modern medicine holds that cancer mortality mainly depends on the structural strength against injury. But this is only part of the factors aggravating the disease and has no overall effect on mortality. Metabolism is the basis for the existence of life. Cancer mortality is the result of equivalent and mutual constraint between functional decline and structural damage. Recovery therapy may prevent the overall lethal effect. The patients may be seriously ill but will not perish. Xu Lingyi, a physician in the Qing dynasty, ever wrote in his book Medical Origins “If the vitality does not get hurt, a serious disease will not result in death. If the vitality does get hurt, a minor disease will result in death”. Modern medicine lacks the equivalent recovery therapy, which is the root cause of most cancer deaths.

Modern medicine cannot prevent or eliminate cancer spread and metastasis. As a result, the therapists stop providing substantial treatment to patients with advanced cancer within one year or so after surgery, radiotherapy and chemotherapy. This makes 90% of patients with advanced cancer die from cancer spread and metastasis. Death is the result of interaction between structural lesion at system level and functional decline at body level. The pathological schematic in Patent Specification shows that advanced cancer patients with cancer spread and metastasis have lesions at both organ level and system level. At this time, there is no functional decline at body level calling for rescue. Timely treatment of the root cause may help the patients to recover, without any possibility death. Death is brought by giving up treatment. The present invention can eliminate the spread and metastasis and significantly reduce mortality.

### (5). On Principles and Methods of Treatment of Major Diseases Proposed in the Patent Specification

Medical advances, in essence, is the development of basic medical science. In accordance with the law of nature, interaction between symmetrical parts will produce changes in the overall nature of things. Principles and methods of treatment of major diseases originate from an overall medical model based on a combination of modern science and modern philosophy which develops from Chinese medicine theory and ancient philosophy. Through holistic treatment of the root cause in a symmetric and equivalent way, the invention cures major diseases and makes major innovations such as Disease Origin and Development Schematic, Schematic for Principles and Methods of Treatment of Major Diseases, and the Introduction of Diseases. It is essentially different from the modern science and experience-based medicine which is structure dominated in the treatment of major diseases and lacks philosophical foundation.

In summary, the core theory of medicine—pathology and introduction to disease are general understanding of the nature of disease which determines the principles of prevention and treatment of disease. Current technology (including traditional medicine and modern medicine) cannot produce a significant effect on the treatment of cancer, suggesting that there is a one-sided understanding of the nature of disease. Otherwise, the hard-to-cures shall not be so prevailing. So to cure cancer, one must comprehensively and profoundly understand the law of disease occurrence and development, understand the nature of cancer, and make theoretical and technological innovations.

### 2. On Cancer Origin and Development Schematic and Schematic for Principles and Methods of Treatment of Major Diseases

(1) Cancer Origin and Development Schematic takes the elements, compounds, cells, tissues and organs, systems, and other organisms at all levels as the main theme. It integrates structure and function and the whole and the local. They are in mutual interdependence and interact with one another, forming an organic whole which is the basis of all life activities. For interaction at the same level, it is a gradual and quantitative process in which the local structure determines the local function. For interaction at different levels, it is a mutational and qualitative process involving the local structure and overall functions in a symmetric and equivalent way. The quantitative change at the same level and the qualitative change at different levels are as important. The two are a unity of opposites. Quantitative and qualitative changes are two forms in the same process. Everything in the world is in a unity of opposites. The symmetry concept applies to all natural phenomena, from the genesis of the universe to each sub-microscopic nuclear reaction process. The basic laws of nature are all in symmetry In Cancer Origin and Development Schematic, all structures and functions interact and combine in a symmetric way. Regardless of whether it is a combination of local functions or surrounding functions, or whether they are in a major-minor relation or an equivalent relation, as long as the level and location of the lesions have been defined in symmetrical combination, one can determine the direction of the proper treatment of the root cause and improve the therapeutic effect. In nature, every living system is a symmetric unity of structure and function, internal factors and external factors, and yin and yang and is indivisible. In Cancer Origin and Development Schematic, one sees the relationship between the overall functional decline and the local structural lesions. The internal and local structural lesions have their own separate identity and location. The same is true with the external and overall functional decline. The objective difference between the two has been revealed. The same is true with their connections. It may lead to the creation of a new theoretical system for pathology.

(2) As is shown in Schematic for Principles and Methods of Treatment of Major Diseases, holistic treatment of the root cause and advanced treatment techniques are all at a higher level than the existing technology with a significant change in the nature. This is also the basis to cure cancer spread and metastasis. The new technology does not rely on any drug or medical device. Human life movement is a unified and harmonious whole integrating all forms of natural movement and is an indivisible advanced form of movement. Separation is a primary form of movement which is not suitable for life. In the therapy of cancer spread and metastasis, mechanical and physical therapy techniques and chemical treatment technologies all follow the laws of nature and are all a unity of opposites. They are unlike the forms in the inanimate natural system which subject to the higher level of life movement. As is shown in Schematic for Principles and Methods of Treatment of Major Diseases, all treatment options and treatment techniques have been combined in accordance with the law of symmetric unity, regardless of whether this combination will produce changes in the number of primary and secondary interactions, or changes in the nature of equivalent interaction. As both sides of the overall unity of opposites are equal, the better the combined symmetry is, the stronger the interaction will be and the more significant the treatment effect will be. In the Schematic, each of the treatment options and treatment technologies can find their place. Any single option or technique can be used for the corresponding lesion. Or they may be used in combination to improve the level of treatment. Advanced treatment technologies include technical quantity changes brought by the sum of treatment factors and technical quality changes brought by the symmetric combination of treatment factors. “Technology breakthrough is the result of the reorganization of the current technologies. In the broadest sense, it is the result of thousands of relatively independent technologies evolving and combining with one another”. Reference News, Nov. 4, 2009, Who leads the social progress: science or technology? By Brian Arthur of the US.

3. On the structure and function of therapeutic device against cancer spread and metastasis The present invention consists of mechanical & physical and chemical components in symmetrical arrangement. It is characterized by the interdependence and interaction between the two components. The overall force generated by mutual reinforcement is used to treat the disease. The invention will not work if it lacks any one of the components. The present invention is mainly used for recovery therapy and also for structural anti-injury treatment to kill cancer cells. The present invention is a combined and interdependent unity of handle, switch, motor housing, multi-functional friction head, ultrasound wave therapy device, laser therapy device, microwave therapy device, RF therapy device, DC low and mid-frequency therapy, chemotherapy, plant drugs, trans-dermal medicine, injection, and intervention therapy, which cannot be separated. Except the multi-functional friction heads, the others are prior arts. Cancer spread and metastasis are characterized by wide range and fast attack. The therapists need to increase both the range and the intensity of treatment. The principles of treatment for the current Cancer Therapy Machine lack the understanding of the position and level of structural and functional lesions during cancer spread and metastasis. They also lack mechanical massage intensity and laser treatment. They cannot meet the treatment needs of cancer spread and metastasis. The present invention has improved the deficiencies of the prior art. Features of therapeutic device against cancer spread and metastasis are described below.

### (1) Mechanical Massage

The main function of human body is composed of mechanical and thermal energy. Mechanical massage and physical sound, light, electricity, magnetism, and heat transfer are two ways to supply energy to restore body function. Both are energy transfer processes and can cause body function changes. They are equivalent in restoring body functions. The difference is that there is no energy transformation in the process of heat transfer. With the mechanical work equivalent to heat transfer, the force causes displacement of the object. The energy transfer process contains a form of energy conversion (e.g. the mutual transformation of mechanical energy and the human body functions). Chemical and botanical drugs can be fully absorbed only by heat transfer and mechanical work. Mechanical movement is the basis for physical and chemical movements interact to transform into the advanced form of life movement. Physical therapy and chemotherapy in the lack of mechanical movement is a primary form of treatment. It cannot achieve the goal of stopping cancer spread and metastasis. Therefore, improving and enhancing the mechanical massage therapy is an essential part in the treatment of cancer.

Among the specific steps in the treatment by the current Cancer Therapy Machine, the first step is shallow trans-dermal administration and frictional heat treatment, followed by drug absorption and deep massage treatment. But the shallow squeezing friction head and deep static friction head are mounted on a common working plane. When the shallow massage is needed, half of the static friction head is doing deep massage. When deep massage is needed, half of the extrusion friction head is doing shallow massage. The treatment cannot be done in accordance with required speed and pressure, making it hard to provide treatment in the correct sequence. As a result, mutually restrained and insufficient therapeutic force reduces the efficacy of the treatment. It cannot be combined with physical therapy to produce an overall mechanical and physical force in a symmetric and equivalent way. It also cannot be formed integrally with the chemical treatment to obtain a holistic approach.

The present invention has integrated the DC squeezing friction head and low and mid-frequency static friction head in therapeutic device against cancer spread and metastasis into a multi-functional friction head. Shallow and deep treatments are applied through adjusting the motor speed in the housing with a switch and adjusting the pressure with a handle. Drug cloth and its clamps are no longer used. It is characterized that the height and position of the round salient at the top of static friction head remain the same. There are two open-type squeezing and blade-like salients with acuate acute angle from squeezing friction head connecting to the round salient. Its height remains the same at the connection with the round salient of the static friction head and gradually reduced from the connection to the margin of the plane where the height becomes the same. Its length is extended from the connection to the margin of the plane of the multi-functional friction head. In prior art, DC therapy is combined with the squeezing and cutting blade-like salient. And low and mid-frequency therapy is combined with the round salient of the static friction head. The motor installed in the motor housing rotates at 300 rpm for shallow massage while the friction head presses the skin with a pressure of 0.5 kg/cm2. Since the muscle and skin has elasticity, the skin depresses for 0.5 cm-1 cm under pressure. The sharp corner and cutting blade-like salient in the multi-functional friction head squeeze the skin with a surface sliding friction, which generates shallow friction heat and gives trans-dermal administration. The force can achieve the acupuncture points and muscles in human body. Friction heat may raise the surface temperatures to 40° C.-48° C., which meets requirements for thermal transfer and drug administration. Working time is fixed to be 3 minutes for local positions. Drugs used are massage oil and safflower oil. As the rotation speed has been reduced by 5 times from 1500 rpm and the skin is in direct contact with lubricants, the cloth used to protect the skin and cloth clamps are no longer necessary. So there is no cloth protection. In deep treatment, the rotation speed is 40 rpm and the pressure increases to 3-5 kg/cm2. As a result, there is more skin and muscle depression. If the pressure increases by 5 times and the speed reduces by 8 times, the friction head and skin surface attract each other to generate static friction force which can reach deep tissue and bone surface. Deep rubbing and friction heat may absorb drugs input by injection and trans-dermal administration. Working time is fixed to be 3 minutes for local positions.

Compared with the Prior Art:

A. 2-50 multi-functional friction heads can be installed in the present invention. They simultaneously provide squeezing friction and then provide static friction. As the treatment force is concentrated, one may determine the rotation speed and pressure according to his needs, thus significantly increasing the treatment effect. The massage may also be provided in therapeutic order, ensuring the quantity of trans-dermal administration and injection applications and thus significantly improving the efficacy.

B. The present invention does not require protective drug cloth or cloth clamps. As rotation speed gets significantly lower, the experiment has proved that the removal of the drug cloth will not damage the skin. After the removal of the drug cloth, the squeezing friction head gets in direct contact with the skin. However, the frictional heating temperature and the effect of trans-dermal drug administration do not get reduced, maintaining all the original functions. Thus, some product elements have been omitted.

### (2). Physical Therapy and Laser Therapy

Cancer spread and metastasis is the greatest threat to human life. The therapists have been trying various technologies to increase the intensity of treatment. Among the existing physical therapies involving heat transfer and function recovery, there are laser therapy, ultrasound heating, radio-frequency ablation, and microwave heating. While restoring function through heat transfer, the technology may also provide anti-injury treatment to kill the cancer cells. Thus it can assist with anti-injury treatment. As various physical heating techniques are fairly slow in taking effect, they can be carried out simultaneously with the shallow treatment. Due to synergistic therapeutic effect, treatment time can be shortened to 10 minutes. The heating temperature for function recovery is generally 40° C.-50° C. And the heating temperature to kill cancer cells is generally 60° C.-100° C. Physical therapies are targeted deep heating treatment. If combined with shallow functional recovery, it can significantly improve the strength and depth of the heating. Otherwise, constrained by the shallow functional decline, deep physical heating only has limited effect. Laser treatment may have the huge energy concentrated in a small area and can be transmitted very far without changing direction. Unlike radiotherapy and chemotherapy, it has no impact on the body. And the treatment can be repeated. Laser prevent cancer cell proliferation. Reference News, Feb. 13, 2013, New Scientist website, February 11. “Laser allows drugs to produce an oxygen structure which destroys cancer cells. Through applying photo-dynamic therapy on adjacent lymphatic vessels, it can help prevent the spread of cancer on its route. And it also destroys the nearby lymphatic vessels.” So the laser in the treatment of cancer plays a unique role. The overall effect of the treatment will be significant. But the current combination of therapies lacks the laser treatment. It cannot meet the needs of treating cancer spread and metastasis. The present invention has combined laser treatment with other physical therapies and has significantly improved the overall effect of physical therapy.

### 4. Treating Functional Decline at System Level During Cancer Spread and Metastasis

In Cancer Origin and Development Schematic, one can see that cancer spread and metastasis is caused by the symmetric and mutual restraint between functional decline at system level and structural lesion at system level. It is an overall lesion of interdependent factors at the same level. The two sides play a primary and secondary role respectively in their efforts. Without treating functional lesions, the treatment of structural lesions will be substantially meaningless. Functional lesions at system level will evolve at a higher pace towards functional lesions at body level which suggests the approach of dying stage. At this stage, one can only alleviate the symptoms by palliative treatment. So the current principle is to increase the intensity of anti-injury treatment to kill cancer cells, supplemented by the treatment of functional lesions. The two sides take effect in a symmetric and mutually reinforcing way and cannot be separated from each other. Otherwise, the treatment will fail. The site with functional decline at system level during cancer spread and metastasis has micro-circulation disturbance, resulting in tissue and organ ischemia, hypoxia and lack of nutrition. This is why it's difficult to cure cancer lesions. As the power of micro-circulation comes from mechanical movement of the surrounding muscle tissues, in terms of mechanical properties, capillaries and the surrounding tissue should be viewed as a whole. When the surrounding muscle tissue stiffens, micro-circulation disturbance thus comes into being. Treatment simply by physical factors such as laser, ultrasonic wave, and microwave, RF heating and blood-activating drugs has only limited effect. Only mechanical massage can relax stiffened muscles, speeding up blood flow by 10 times. Supplemented with physical heating and blood-activating drugs, it can restore micro-circulation and alleviate ischemia, hypoxia, and lack of nutrition. Meanwhile, at the functional lesions at system level, there also exist a small number of cancer cells. There are the following two causes: First, there already exist a small number of cancer cells at functional lesions before treatment. Second, during anti-injury treatment and function recovery, mechanical massage, extrusion, hot compress and physical therapy promote cancer cells to fall off. Therefore, the principle of treating functional decline around cancer lesion at system level is that, during recovery-based treatment, it's necessary to provide anti-injury treatment to kill cancer cells. Thus guarantee recovery in a number of lesions without cancer spread and metastasis. In overall treatment of cancer spread and metastasis, recovering function around the lesions and anti-injury treatment to kill cancer cells are mutually reinforcing and conducted at the same time. The specific treatment principles are as follows.

(1) At functional decline lesion at the system level, shallow treatment shall be first provided. Coat the treated area with blood activating drugs such as massage oil, safflower oil, nitroglycerin, or Curcuma oil according to the disease. Blood activating drugs can promote blood circulation at lesions, increase the oxygen content in the blood, relieve ischemic conditions, and increase the sensitivity of tumor tissue to radiation or chemotherapy. They also have anti-inflammatory and anti-inflammatory effects. Trans-dermal administration is different from skin agents in their effects. Trans-dermal administration in shallow treatment targets the whole body and deep tissue. In therapy device, DC conductor is connected to the squeezing metal and sharp corner cutting edge salients. Pulsed DC current provides treatment through the salients. DC current treatment expands blood vessels, promote blood circulation and trans-dermal drug administration. It also promotes local tissue metabolism and has antibacterial effect. Squeezing drug massage is appropriate for friction heating on skin surface and trans-dermal drug administration. Although the two methods follow different principles, the treatment site level and therapeutic effect are the same. Combining drug massage, trans-dermal drug administration, and DC current therapy for shallow treatment will significantly improve the treatment effects. Same with function recovery at deep level, the patients do not need to consume their body's energy and the consumption of mechanical, thermal, electrical, and chemical energy get replenished directly. This alleviates energy missing at lesions and also promotes functional recovery at deep level. Shallow level refers to the skin and muscle level. Local treatment time at shallow level is 3 minutes.

(2) Complete functional recovery at deep level after functional recovery at shallow level. Deep and shallow recovery belongs to a symmetric overall treatment which can significantly improve the effects. Deep level refers to the deep tissue in human body and bone surface. Local treatment time is also 3 minutes. First, conduct intramuscular injection, acupoint injection, intravenous injection, needle-free high-pressure injection, and iontophoresis administration. Blood activating and anti-cancer drugs include Rhizoma Chuanxiong Safflower Injection and Salvia Injection. Anti-cancer drugs include Aconite Injection, Brucea Injection, Ginseng Polysacchride Injection and 5-fluorouracil. Static friction is appropriate for deep tissue and muscle massage. Internal heat generated when relaxing muscles, combined with drugs, promotes blood circulation and drug absorption and utilization. The main effect of low and middle pulse current treatment is to excite nerves and treat nerve and muscle disease. In low and middle pulse current treatment, the electrode plates are made into eccentric, round and projected static friction heads which are connected with the conductor. Low and middle pulse current treatment excites nerves. Static friction massage relaxes the muscles and absorb drugs. These two has been combined into an overall treatment. Meanwhile, static friction head can also be made into metal shapes and combined with pulsed DC current.

In functional recovery at deep level, heat transfer technologies in the present invention, such as laser, ultrasonic wave, microwave, and radio frequency, are precise, deep penetrating, and controllable in heat transfer and function recovery. As the treatment takes a long time, it may start at the same time with shallow treatment and give heating treatment to lesions. In treatment, it combines with static friction and mechanical work and becomes a whole in a symmetric and equivalent way. They work together at the lesions. It combines with chemical drugs and form an advanced treatment technique which is an essential factor in function recovery in cancer treatment. Without it, it becomes impractical to conduct functional recovery at deep level. Advanced treatment techniques can be combined into an overall treatment. Cancer treatment will become a partial treatment which cannot cure cancer. Meanwhile, for overall treatment involving anti-injury and function recovery, one shall not only complete the current anti-injury treatment testing standards, but also adopt double standards to check function recovery.

### 5. Prevent Cancer Spread and Metastasis by Treating the Root Cause

Renowned cancer expert Professor He Yumin claimed that “preventing spread, metastasis and relapse is the key to tumor treatment. For that, modern medicine almost has no good approach. This is precisely where Chinese traditional medicine enjoys advantages.” So how to prevent cancer spread and metastasis? One should find the root cause and provide treatment accordingly. They are described as follows.

(1) Cause of cancer spread and metastasis In Cancer Origin and Development Schematic, one can see that cancer spread and metastasis is caused by the symmetric and mutual restraint between functional decline at system level and structural lesion at system level. It develops from mutual restraint of structural lesions at organ level and functional lesions at system level of the advanced cancer. As functions at system level around the primary lesions of the cancer cell at the organ level decline in a symmetric and equivalent way, they are in a state of ischemia, anoxia, and micro-circulation block and contain a small number of cancer cells. As the two sides are at different levels and change in a symmetric and equivalent way, if the therapy does not provide an overall treatment for symmetric and equivalent structures and functions, the primary lesions in the structures at organ level will spread and transfer to system level, leading to disease deterioration. Modern medicine holds that systemic functional lesions have taken place, but cannot determine the specific location of the lesions. Thus it lacks an equivalent treatment for functions around the lesions of cancer cells. This is the real cause of cancer spread and metastasis. Therefore, the overall treatment of the root cause for advanced cancer is to prevent spread and metastasis. The two sides is in a causal relationship and are two aspects of the same issue. Spread and metastasis is the inevitable result of symptomatic treatment involving chemotherapy and surgery.

### (2) Treatment Preventing Cancer Spread and Metastasis

Chinese traditional medicine follows the principle of strengthening body resistance and eliminating evil, treating both manifestation and root cause of disease, and a balance of yin and yang. Treatment preventing cancer spread and metastasis refers to the application of the present invention to provide symmetric and equivalent recovery treatment for functional lesions at system level around the primary lesions of the cancer cell while surgery and chemotherapy-based treatment is provided for the primary lesions of the cancer cell at organ level. The recovery treatment may also eliminate the side effects of chemotherapy and surgery. The overall and mutually reinforcing treatment may kill cancer cells, eliminate the primary lesion, and prevent cancer spread and metastasis. Specific operation is as follows: (1) First, use prior art to determine the treatment site. Provide surgery and chemotherapy-based treatment for the primary lesions of the cancer cell. Use the present invention to provide mechanical, physical, and chemical treatment for the primary lesions of the cancer cell for 10 minutes to restore the functions. Refer to the patent specification for specific operational requirements. (2) Procedure and intensity of the overall treatment: In general, patients in good physical health receive chemotherapy and surgery before recovery treatment. Weak patients shall receive recovery treatment before chemotherapy and surgery. The procedure varies from person to person. If the patients need to increase the intensity of recovery treatment, they may increase the number of treatment or expand the range of treatment. (3) Determine the time of treatment: As structural and functional lesions are symmetrical and equivalent at different levels, they have a mutational nature. It is generally accepted that overall symmetrical and equivalent treatment shall be done once a day for one month. According to the patient's physical condition, the remaining time shall be spent on overall treatment to kill cancer cells or for recovery. The treatment shall be done with relatively lower intensity. The whole treatment will last 1-3 years. Most patients require long-term recovery-oriented treatment. As the recovery treatment is to eliminate the side effects of radiotherapy and chemotherapy and to restore the physical condition of the patients, no matter whether in mutational or progressive period, there is no side effect. The 50% mortality of patients receiving surgery and chemotherapy will never happen to this treatment. And it will not increase the cost of treatment. When the structural treatment is tested, recovery treatment will receive double standard tests. During 1-3 years of long-term treatment, patients will gradually develop towards a positive state. Spread and metastasis among 90% of patients will never happen.

### 6. Treating Structural Damage at System Level During Cancer Spread and Metastasis

Why did 50% of cancer patients die from a variety of factors during surgery and chemotherapy? Why did 90% of advanced cancer patients exhibit spread and metastasis within a year after surgery, radiotherapy and chemotherapy? Why can't so many technical innovations solve this critical problem fundamentally? This is a question of treatment orientation. This is a philosophical question with universal connotation, not purely technical problems. Structural damage and functional decline in cancer treatment are two sides of a whole. They depend on each other and cannot be separated. The lack of any one side will lead to treatment failure. For the overall treatment, one needs to align the two sides on the lesion at the same time. Surgery and chemotherapy-based treatment lacks an equivalent part, that is, the function recovery treatment for the lesion. This is a treatment with mixed gains and losses. Although it may relieve symptoms, it's detrimental to the survival of patients. The treatment direction is wrong. And this is the fundamental cause of cancer treatment failure which promotes spread and metastasis.

Among lesions involving structural damage at system level, several have changed to lesions involving spread and metastasis. But this is merely a change in number. It is essentially different from the primary lesions at organ level. Functional lesions around the primary lesion are symmetric and equivalent to structure damage. It is difficult to cure. At the same level, functional lesions are not around the structural lesions, but a feature of function itself. Functions around diffused and transferred cancer cell lesions at system level are body-level functions which have not yet developed lesions according to the development order. Functional decline at body level is lethal in nature. Any treatment will be of little help. Therefore, by treating functional decline at system level and increasing the intensity of killing cancer cells, one can quickly stop cancer spread and metastasis. The specific treatment principles are as follows.

(1) Currently in cancer treatment, 70 percent of the malignant tumors need to receive radiation therapy, including pre-surgery and post-surgery radiotherapy, simple radiotherapy, radiotherapy—chemotherapy combined therapy and surgery-radiotherapy-chemotherapy combined therapy. The role of radiation in killing the tumor is the same with chemotherapy drugs. Systemic reactions are lighter than drugs. Local effects are 10-20 times stronger than drugs. However, the radiation does not simply kill cancer cells. It also damages normal cells. The only difference lies in that the normal tissues after being damaged by radiation may restore themselves much more quickly than tumor tissues. Maximize tumor destruction while maximizing the protection of normal tissues. This is a goal which clinical radiotherapy dosimetry and radiotherapy providers have been pursuing for long. In general radiotherapy (X knife and gamma knife excluded), there are more than 30 times of exposure from the beginning to the end. One course may last about 40 days. Each exposure lasts only a few minutes. The basic principle of the radiation therapy of tumors is that the radiation dose is controlled within a certain range and gradually increases the cumulative damage of tumor cells while it is still possible to restore the function of normal cells. To shorten the course of treatment, the dose is bound to be increased. It is possible to kill tumor tissue and normal cells at the same time. Therefore, radiation therapy does not follow the basic principle of unity of opposites. There is no symmetric recovery therapy in this treatment. Balance and stability are in lack. One hopes to maintain long-term stability by controlling the number of the killed cancer cells, so as to achieve treatment goals. This is basically impossible. The results of most radiotherapy patients have proven this point.

Surgery and chemotherapy-based anti-injury treatment and function recovery treatment are opposites in unity. They depend on each other and cannot be separated. Function recovery and surgery and chemotherapy-based anti-injury treatment are equivalent and symmetric. They are mutually reinforcing and cannot be separated from each other. This holistic treatment is present in the whole process treating cancer spread and metastasis. The correct radiation therapy to kill cancer cells should be: 5-10 minutes before radiotherapy, apply DC low and mid-frequency multi-functional friction head, laser, ultrasound wave, microwave, radio frequency, blood activating drugs, and injection at the same time. Provide function recovery treatment for 3-5 minutes. Then give radiation therapy for several minutes. The object is to increase radiation sensitivity and intensity. 5-10 minutes after radiotherapy, apply the present invention to radiotherapy centers to give function recovery treatment for 3-5 minutes. Its main purpose is to help normal cells to restore function. For systemic symptoms and side effects of chemotherapy and surgery, apply the present invention to the whole body to give function recovery treatment. It can eliminate the side effects and symptoms and restore functions. Systemic treatment lasts for 10-15 minutes each time. It needs to be given once a day. 15 times constitute a course of treatment.

In the recovery treatment, as normal cells outnumber cancer cells, they obtain significantly more nutrition and energy. Especially, functions around the spread and metastasis lesions are normal almost without any cancer cells. Normal cells after obtaining energy may restore functions and help the treatment. The effectiveness of radiation therapy depends on whether the tumor is radio-sensitive. Radio-sensitivity in turn depends largely on the oxygen content within the tumor. That which has abundant blood supply is the tumor with high oxygen content. So before and after the radiotherapy, function recovery treatment shall be given to cancer cell lesions and around. This is the correct way to increase the radiation sensitivity, increase the intensity of anti-cancer, reduce the side effects, and restore anti-cancer ability. The nature and structural adjustment of therapeutic factors is equally important as the increase of intensity. They are interdependent and indispensable.

(2) Currently in cancer chemotherapy, the therapists have tried updating chemotherapy drugs, increasing the dose, extending the treatment, or combining various chemotherapy drugs. While the efficacy of a small number of cancers has been improved, the long-term efficacy and five-year survival rate have not been much improved. Drug resistance leads to 90 percent of cancer metastasis treatment ending in failure. Obviously, this is not a question about the chemotherapy technology. Same with surgery and radiotherapy, there is a problem in terms of the treatment orientation. For cancer lesions at the same level, structural lesion and functional lesion are in interdependent relations. Although the intensity difference divides them into primary and secondary side, simply killing cancer cells will surely be subject to resistance from the unity. This will result in side effects and drug resistance, leading to treatment failure. Modern medicine has analyzed the reasons for the failure of chemotherapy and they roughly fall into the following categories:

a. Cancer cell proliferation rate and metabolism are different, even for tumors on the same body.

b. The tumor is in large size with few blood vessels at its center. It is difficult for drugs to take effect. So the drugs on patients with advanced cancer are less effective than on patients with early cancer.

c. The toxicity of chemical drugs also brings damage to normal organs.

d. The tumor cells become resistant to drugs. The sensitive part is eliminated. But the not sensitive part is left to continue breeding. It will make the original drugs ineffective.

e. Chemical drugs on tumor follow the law of “log killing”. The drugs may kill the vast majority of cancer cells, but cannot kill them all. This is why chemotherapy needs to be supplemented by immune therapy or traditional Chinese medicine.

According to the concept of holistic medicine, the more vigorous the cell metabolism is, the more vulnerable it is to chemotherapy attacks. In treatment, the part with normal functions absorb more nutrients. Thus cancer cells are more sensitive and prone to drugs. The part with declined functions absorb less nutrients. Thus cancer cells are less sensitive and prone to drugs. Radiotherapy and chemotherapy kill the cancer cells at the expense of the patients' cancer-fighting ability. The effectiveness of chemotherapy depends on whether the tumor is sensitive to chemotherapy. Therefore, symmetric recovery treatment shall be given to cancer lesions and their vicinity. This is the correct way to increase the radiation sensitivity, increase the intensity of anti-cancer, reduce the side effects, and restore anti-cancer ability. For the lesions, anti-injury treatment supplemented by recovery treatment may eliminate the side effects and help chemotherapy to take effect. For lesion vicinity, recovery treatment supplemented by anti-injury treatment may enhance the body's anti-cancer ability, stop cancer spread and metastasis, and help long-term treatment. The present invention gives recovery treatment to lesions. Under the impact of mechanical massage and ultrasonic vibration, capillary openings per cubic millimeter increase from 31 to 1400. And the flow of lymph speeds up by 7 times. When the temperature at the treatment site rises to 42° C. under the impact of laser, ultrasound wave, microwave, RF and other physical factors, the effect of applying doxorubicin, bleomycin, nitroso urea and other anti-cancer drugs becomes 10-100 times higher than that at 37° C. When supplemented by blood activating drugs, the efficacy will be significantly improved. For current chemotherapy without recovery treatment, administration and subsequent withdrawal period make up a course. The whole treatment normally takes 4-6 courses. The total time required will be several months or even a year or more. An important objective of multi-course chemotherapy is to allow normal cells to restore function and save energy for the next course. This passive waiting and separation significantly reduces the intensity of treatment and misses the best timing of treatment, resulting in limited efficacy of chemotherapy. In the holistic treatment, protection and restoration of normal cell function has been completed during the treatment. Metabolic functions as the basis for patients' life are protected, with no need to wait. The may give a significant effect. And the patients will not die in holistic treatment.

The tumors which have already diffused and transferred tend to lose the chance of surgery or radiotherapy. But chemotherapy may still take effect. Many chemotherapies are provided in prior art. They include: a. Oral administration, b. Intramuscular injection, c. intravenous injection, d. intravenous pulse, e. intravenous infusion, f. arterial injection, g. articular injection, h. intrathecal injection, i. tumor injection, j. local application. One of them is chosen now to show how to make improvements. Specific measures are as follows: Qilu Evening News, Apr. 28, 2007; New Morning, Jul. 8, 2010; Sustained Release Therapy, Inventor: Yu Baofa, Proprietary name: A Composition for Treating Tumors, Patent No. 200610151437.0. “This therapy applies chemical drugs not through the blood. Guided instead by the imaging equipment and through percutaneous puncture, the therapist injects the medicine directly into the interior of the tumor. The injection takes effect in the tumor. Its half-life period extends by scores of times. But it is not involved in the blood circulation. It can be applied to various primary or metastatic solid tumors, including lung cancer, liver cancer, pancreas cancer, esophagus cancer, stomach cancer, colon cancer, prostate cancer, kidney cancer, oral cancer, nasopharyngeal cancer, cervical cancer, ovarian cancer, breast cancer, skin cancer, malignant lymphoma cancer, and bone tumors”. However, simply changing the route of administration is unlikely to significantly improve the effect of cancer chemotherapy. Even though applying embolic agent may improve efficacy, the effect is quite limited.

The holistic treatment converts energy through mechanical work, transfers heat by physical therapy, and improves micro-circulation by applying blood activating drugs. It may eliminate the side effects and significantly increase the intensity of chemotherapy. The specific method is to give recovery treatment to tumors for 8-10 minutes after injection of chemical drugs. They can be done on the same site at the same time. But the intensity of recovery treatment is significantly less than that of the chemical drugs because it is a symmetric holistic treatment. For treatment at the same level, recovery treatment shall be simultaneously given regardless of the specific chemotherapy, to ensure that the treatment is in the right direction.

(3) Surgery is not combined with the symmetric recovery treatment. This goes against the holistic treatment principle. The treatment is in the wrong direction, with no possibility of achieving therapeutic purposes. This is the root cause for recurrence of spread and metastasis after surgical treatment. Killing the cancer cells does not mean the increase of normal cells. One shall forfeit the hope of curing cancer with just one surgery. Meanwhile, the intensity and range of surgery shall be reduced. Instead, the intensity and range of preoperative and postoperative recovery treatment shall be determined in accordance with the principle of holistic treatment. Surgery and anti-injury treatment shall be combined with the recovery treatment in a symmetric, equivalent and mutually reinforcing way to form a holistic treatment which goes in the right direction and achieves therapeutic purposes. Mechanical massage, physical heating, and chemical energy supplement in the present invention can be applied in recovery treatment for the lesions before and after surgery. As the present invention is able to kill cancer cells, it can be applied separately around the lesion before cancer spread and metastasis to restore function and prevent spread and metastasis. For lesions where spread and metastasis have already begun, the present invention shall be applied along with surgery, radiotherapy and chemotherapy. They cannot be separately applied. Treatment of other configurations may also achieve the object of the present invention.

### 7. Summary

Modern philosophy combined with modern science and technology is the right way to cure cancer. But in the present it's hard to reach any consensus. The DNA double helix discoverer, James Watson, ever wrote in his report that “I'm interested in DNA just because unlocking the genetic code may help cure certain diseases. I'm not interested in religion and philosophy because they are too remote”. (See Science Times, Page A2, Apr. 14, 2010) In this regard, the Minister of Health, Chen Zhu, wrote in the foreword to Cancer: Evolutionary Legacy, “Scientists constantly make new breakthroughs in the microscopic study of cancer gene. But there is still no unified and macroscopic understanding”. Renowned expert Professor He Yumin believes that restoring in vivo metabolism environment is the real cure for cancer. It will open up a hopeful shortcut for human to defeat cancer. In the patent specification of therapeutic device against cancer spread and metastasis, the inventor proposed a major principle and method of cancer treatment. This was a holistic treatment combining modern science with modern philosophy. It made use of advanced treatment techniques to restore in vivo metabolism environment and, meanwhile, to kill cancer cells to cure cancer. Based on this theory and technology, the present invention has made improvement in treating cancer spread and metastasis. The inventor hopes to make efforts to cure cancer spread and metastasis and brings hopes to patients who are currently incurable.

### DESCRIPTION OF FIGURES

FIG. 1: Front view of the therapeutic device against cancer spread and metastasis,

FIG. 2: A-direction view of multi-functional friction head in therapeutic device,

FIG. 3: B-direction view of multi-functional friction head in therapeutic device,

FIG. 4: Cancer origin and development schematic,

FIG. 5: Schematic for principles and methods of treatment of cancers.

### SPECIFIC MODE FOR CARRYING OUT THE INVENTION

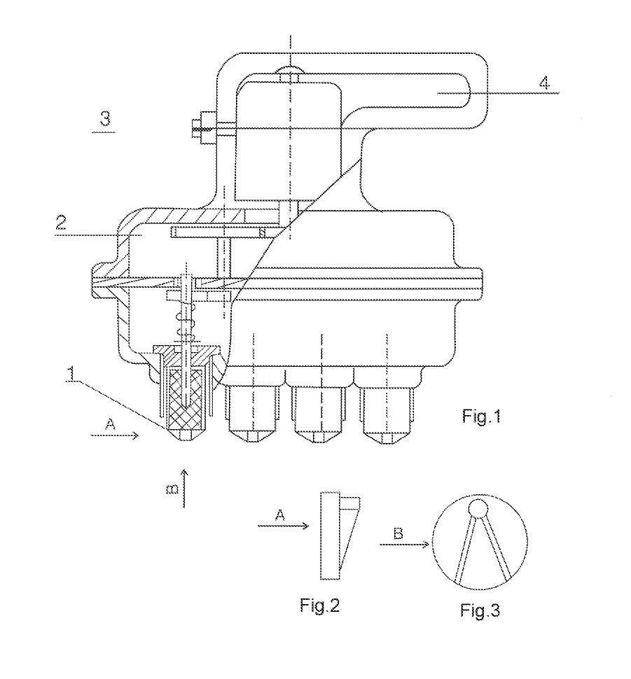

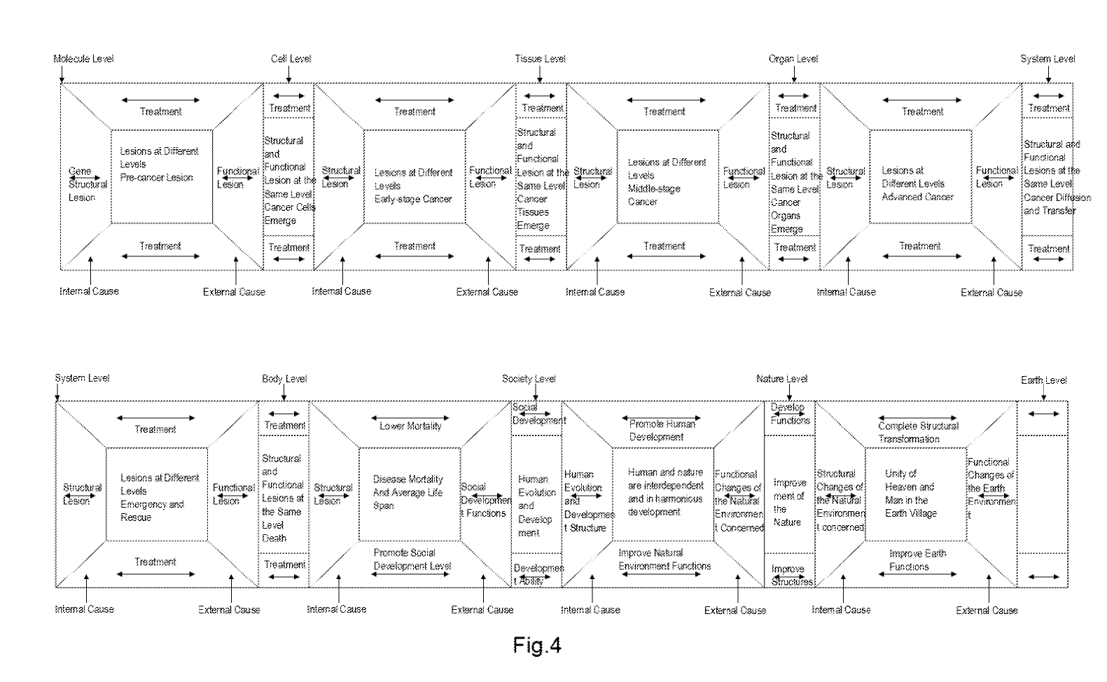

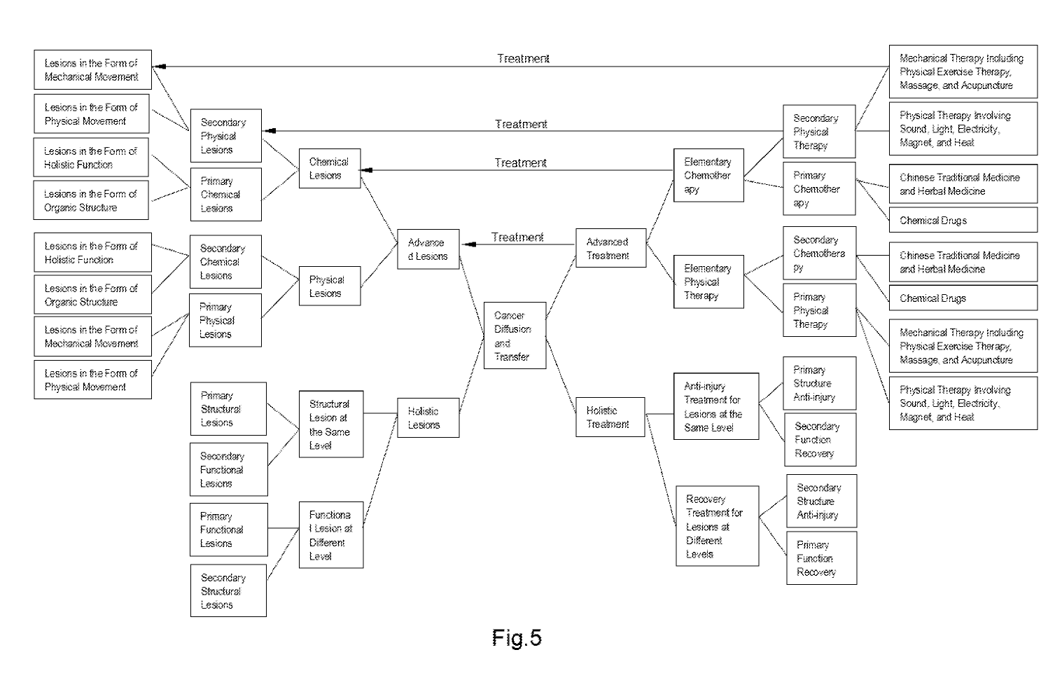

In FIG. 1: 1 is the multi-functional friction head; 2 is the motor housing; 3 is the switch; and 4 is the handle. FIG. 2 is A-direction View of the Multi-functional Friction Head. FIG. 3 is B-direction View of the Multi-functional Friction Head.

The present invention is achieved by the following technical solutions: A therapeutic device against cancer spread and metastasis, is made up of the DC low and mid-frequency multi-functional friction head, the motor housing, the switch, and the handle, combining with the laser, the ultrasound wave, the microwave, the RF, and chemical drugs to work together. It gives recovery treatment to lesions with cancer spread and metastasis, supplemented by treatment killing cancer cells. It combines with surgery and chemotherapy in a symmetric, equivalent and mutually reinforcing way to form a holistic treatment to cure cancer spread and metastasis. In the multi-functional friction head (1), the static friction head and squeezing friction head are combined into one with multiple functions of static friction and squeezing friction. The round salient at the top of the static friction head is connected to the joint of two acuate acute angle shape salients which are arranged in a fan-shaped angle on the squeezing friction head. The length of the squeezing friction head is from the connection with the static friction head to the far edge of the plane of the multi-functional friction head (1). Its height remains the same at the connection with the round salient of the static friction head and gradually reduces from the connection to the margin of the plane where the height becomes the same. The multi-functional friction head (1) is installed in parallel in the motor housing (2) and in contact with the skin. Turn on the switch (3) to start high-speed rotation. Hold the handle (4) and gently press to initiate squeezing friction. Low-speed rotation and heavy pressure initiate static friction. The multi-functional friction head (1), and the laser treatment, the ultrasound wave treatment, the microwave treatment, the RF treatment and the chemical drugs work together to form a holistic treatment system, which cannot be separated.

Treatment steps are as follows:

1. For treatment on shallow parts, coat the skin with nutrients, vitamins, blood activating drugs and anti-cancer drugs. Turn on the switch and start DC electrotherapy, drug squeezing massage, and trans-dermal drug administration. Treatment for each site lasts 3 minutes. Multiple sites can be treated simultaneously. Then start treatment on deep parts. Static friction massage relaxes the muscles, generating heat and absorbing drugs. Low and mid-frequency electric treatment stimulates the nerve and generates heat. Local injection of nutrients, blood activating drugs and anti-cancer drugs. Various treatments are jointly applied. Treatment on each site lasts 5 minutes. Drug injection, intervention, laser, ultrasonic wave, microwave, and radio frequency treatment for deep parts are combined with treatments for shallow parts. The treatment lasts 10-15 minutes. The intensity and time of all treatment factors can be adjusted according to the patient's condition and treatment requirements. Recovery treatment is an adaptive stimulation which shall make the patient feel comfortable and receptive.

2. Once a day and 15 times per course of treatment. It is alternately applied with chemotherapy and surgery. The two treatments are interconnected and cannot break apart. The treatment can be carried out before or after surgery and chemotherapy with an interval of 2-5 minutes. The treatment is determined depending on the patient's condition.

3. Increase the rotation speed of the friction head. Increase the local trans-dermal administration and injection. Increased muscle tissue depth in massage and the intensity of DC low and mid-frequency electric therapy, laser, ultrasonic, microwave, and radio frequency, anti-cancer drugs and therapeutic device against cancer spread and metastasis can also kill cancer cells. When surgery and chemotherapy are not effective in killing cancer cells, the present invention can be used to give anti-injury treatment and to kill cancer cells. Anti-injury treatment is a non-adaptive stimulation therapy. Patients do not feel comfortable.

4. For feeble patients who cannot receive surgery or high-dose chemotherapy, the recovery treatment of the present invention can be provided to supplement surgery and chemotherapy. For oral drugs, infusion, nutrients, and fitness activities in adjuvant systemic therapy, they can be carried out according to the condition of the patients.

### Embodiments of the Present Invention

Apply mechanical drug massage provided by the therapeutic device against cancer spread and metastasis. It combines the squeezing friction head with drug coating. Its nature is the same with an ancient Chinese medicine—Cream Massage. The instrument has a speed control switch and 4 squeezing friction heads. The diameter of the friction head is 1.5 cm and the rotation speed is 500 rpm. The patients' treatment site is coated with massage oil, safflower oil, and blood activating drugs. The product has been approved by the Food and Drug Administration of Shandong Province. The product registration number is Lu. Drug Admin. Equip. (Test) 2000 No. 2020044. The product has been in clinical use in Qingdao Municipal Hospital, Qingdao Hospital of Traditional Chinese Medicine, Qingdao Central Hospital, Children's Hospital of Qingdao, and the Third People's Hospital of Qingdao for many years. The treatment covers diabetes, cerebrovascular disease, and coronary heart disease. It's welcomed by patients. In Qingdao Tumor Hospital, doctors there operated the device to treat tumor lesions and the adjacent parts and acupoints. Each treatment lasted 15-15 minutes and was given once a day. 15 times constituted a course of treatment. Many cancer patients with spread and metastasis suffered from physical deterioration, nausea, hair loss, and loss of appetite after receiving surgery and radiotherapy. Through recovery treatment and drug massage, the suffering of patents got relieved and the quality of life got improved. They were more willing to accept chemotherapy with a good effect. Patients are generally willing to receive treatment by the device and hope to operate the device by themselves.

1. A therapeutic device against cancer spread and metastasis, is made up of the DC low and mid-frequency multi-functional friction head, the motor housing, the switch and the handle, combining with the laser, the ultrasound wave, the microwave, the radio frequency and chemical drugs to work together. It gives recovery treatment to lesions with cancer spread and metastasis, supplemented by treatment killing cancer cells. It combines with surgery and chemotherapy in a symmetric, equivalent and mutually reinforcing way to form a holistic treatment to cure cancer spread and metastasis. In the multi-functional friction head (1), the static friction head and squeezing friction head are combined into one with multiple functions of static friction and squeezing friction. The round salient at the top of the static friction head is connected to the joint of two acuate acute angle shape salients which are arranged in a fan-shaped angle on the squeezing friction head. The length of the squeezing friction head is from the connection with the static friction head to the far edge of the plane of the multi-functional friction head (1). Its height remains the same at the connection with the round salient of the static friction head and gradually reduces from the connection to the margin of the plane where the height becomes the same. The multi-functional friction head (1) is installed in parallel in the motor housing (2) and in contact with the skin. Turn on the switch (3) to start high-speed rotation. Hold the handle (4) and gently press to initiate squeezing friction. Low-speed rotation and heavy pressure initiate static friction. The multi-functional friction head (1), and the laser treatment, the ultrasound wave treatment, the microwave treatment, the RF treatment and the chemical drugs work together to form a holistic treatment system, which cannot be separated.
